(19) United States
(12) Patent Application Publication
(10) Pub. No.: US 2014/0180369 A1
(43) Pub. Date: Jun. 26, 2014

(54) CONTINUOUS THERMOTHERAPY DEVICE

(71) Applicants: Seo Jun Oh, Seoul (KP); Seo Min Oh, Seoul (KP)

(72) Inventors: Seo Jun Oh, Seoul (KP); Seo Min Oh, Seoul (KP)

(21) Appl. No.: 14/238,048

(22) PCT Filed: Aug. 10, 2012
(86) PCT No.: PCT/KR12/06382

§ 371 Date: Feb. 10, 2014

(30) Foreign Application Priority Data
Aug. 10, 2011 (KP) 10-2011-0079864
Nov. 9, 2011 (KP) 10-2011-0116630

(51) Int. Cl. A61F 7/03

(52) U.S. Cl. CPC A61F 7/03
USPC 607/112

(57) ABSTRACT

The present invention relates to a continuous thermotherapy device, and comprises: a belt; a receiving box which is provided on the belt; a hot pack case which is received by the receiving box; and a hot pack which is inserted into the hot pack case.

### TECHNICAL FIELD

The present invention relates to a continuous thermotherapy device, and more particularly, to a continuous thermotherapy device which can stably maintain temperature of a hot pack suitable for human bodies for a long period of time, by adjusting an amount of air contacting the hot pack.

### BACKGROUND

Physiologically, the human abdomen is a very important body part that must always be warmly protected for organ activation within an abdominal cavity, blood circulation, digestion promotion of food, and the like.

Many persons are subjected to thermotherapy massage on an abdominal portion with a hot pack, causing exothermic reaction, for healthy blood circulation as well as treatment of various abdominal problems such as abdominal obesity, a digestive disorder, evacuation disorder, micturition disorder, hemorrhoids, malfunction of various organs, a pain of various body parts, a stitch of the waist, and the like.

A commercially available hot pack is heated up to a temperature ranging from 70° C. to 80° C. within several minutes after a vinyl pouch for storing the hot pack is open and the hot pack is properly shaken in the air such that contents in the hot pack are mixed. Thereafter, after a period of time from 12 hours to 15 hours, the temperature of the hot pack rapidly decreases, and after about 20 hours, the hot pack cools down. The hot pack heated up to a temperature of 70° C. or higher in an initial stage is so hot that the skin can be burned, and a significant number of hot pack users actually experience a burn to a degree of causing a blister (a second degree burn).

Hot packs sold in the market have a considerable empty space therein such that contents thereof can be easily mixed to facilitate exothermic reaction. If the hot pack is worn on a body part parallel to gravity such as the abdomen, the contents of the hot pack are gathered at a lower portion thereof to be conglomerated over time. The conglomerated contents are in an unstable state in whichin there is a great difference in temperature according to a portion, in which only the bulgiest portion is excessively pressed while being attached to the skin of the abdomen, whereby a heat transfer surface becomes extremely narrow and thus a risk of a burn on the attached portion is greatly increased.

Due to such reasons, users avoid thermotherapy massage on the abdomen with the hot pack, although it is known that warming the abdomen through the hot pack makes users feel better and is good for health.

Therefore, there is a need to solve such problems.

### SUMMARY

The present invention has been made to solve such problems in the art and an aspect of the present invention is to provide a continuous thermotherapy device which can stably maintain temperature of a hot pack suitable for human bodies for a long period of time, by adjusting an amount of air contacting the hot pack.

In accordance with one aspect of the present invention, a continuous thermotherapy device includes: a belt; a receiving box provided to the belt; a hot pack case received in the receiving box; and a hot pack inserted into the hot pack case.

The hot pack may include: a pyrogen; and a housing receiving the pyrogen therein and having an internal space larger than a volume of the pyrogen such that the pyrogen is mixed within the internal space.

The housing may include: a front plate; a rear plate having the same shape as the front plate and stitched at upper and lower ends thereof to upper and lower ends of the front plate, respectively; a right plate connected at a front end thereof to the front plate and at a rear end thereof to the rear plate; and a left plate having the same shape as the right plate and facing the right plate, the left plate being connected at a front end thereof to the front plate and at a rear end thereof to the rear plate, wherein a width of each of upper and lower ends of the left and right plates may be smaller than a width of a central portion of each of the left and right plates.

The front and rear plates may be foldable and, when lower portions of the front and rear plates are folded, the width of the lower end of each of the left and right plates may be the same as that of the central portion of each of the left and right plates.

The receiving box may include at least one of an inner receiving box open at an upper side thereof and disposed inside the belt, and an outer receiving box open at an upper side thereof and disposed outside the belt.

Velcro tapes may be provided to an inner surface of an upper end of the receiving box and to one side surface of the belt facing the inner surface of the upper end of the receiving box, respectively, and the upper end of the receiving box may be detachably coupled to the belt through attachment/detachment of the Velcro tapes.

The hot pack case may include a case main body that has an upper opening and is formed in a hexahedral shape, wherein an upper thickness of a side surface of the case main body may be larger than a lower thickness of the side surface of the case main body.

The case main body may be formed with an air contact hole.

The continuous thermotherapy device may further include: an air shielding film surrounding the hot pack such that an amount of air contacting the hot pack is adjusted, wherein the air shielding film may include at least one of a front shielding film surrounding a front surface of the hot pack; a rear shielding film surrounding a rear surface of the hot pack; a top shielding film surrounding a top surface of the hot pack; a bottom shielding film surrounding a bottom surface of the hot pack; a first integral shielding film surrounding the front, bottom and rear surfaces of the hot pack; and a second integral shielding film surrounding the front, top, and rear surfaces of the hot pack.

In accordance with another aspect of the present invention, a continuous thermotherapy device includes: a belt; a receiving box provided to the belt; a hot pack pouch received in the receiving box and formed of a flexible material to enclose a bent portion; and a hot pack inserted into the hot pack pouch.

The hot pack may include: a pyrogen; and a housing receiving the pyrogen therein and having an internal space larger than a volume of the pyrogen such that the pyrogen is mixed within the internal space.

The housing may include: a front plate; a rear plate having the same shape as the front plate and stitched at upper and lower ends thereof to upper and lower ends of the front plate, respectively; a right plate connected at a front end thereof to the front plate and at a rear end thereof to the rear plate; and a left plate having the same shape as the right plate and facing the right plate, the left plate being connected at a front end thereof to the front plate and at a rear end thereof to the rear plate, wherein a width of each of upper and lower ends of the left and right plates may be smaller than a width of a central portion of each of the left and right plates.

In accordance with a further aspect of the present invention, a continuous thermotherapy device includes: a hot pack; and a hot pack case receiving the hot pack, wherein the hot pack case may include a case main body having an upper opening and formed in a hexahedral shape.

The hot pack may include: a pyrogen; and a housing receiving the pyrogen therein and having an internal space larger than a volume of the pyrogen such that the pyrogen is mixed within the internal space.

The housing may include: a front plate; a rear plate having the same shape as the front plate and stitched at upper and lower ends thereof to upper and lower ends of the front plate, respectively; a right plate connected at a front end thereof to the front plate and at a rear end thereof to the rear plate; and a left plate having the same shape as the right plate and facing the right plate, the left plate being connected at a front end thereof to the front plate and at a rear end thereof to the rear plate, wherein a width of each of upper and lower ends of the left and right plates may be smaller than a width of a central portion of each of the left and right plates.

An upper thickness of a side surface of the case main body may be larger than a lower thickness of the side surface of the case main body, and the case main body may be formed with an air contact hole.

The continuous thermotherapy device further includes: an air shielding film surrounding the hot pack such that an amount of air contacting the hot pack is adjusted, wherein the air shielding film may include at least one of a front shielding film surrounding a front surface of the hot pack; a rear shielding film surrounding a rear surface of the hot pack; a top shielding film surrounding a top surface of the hot pack; a bottom shielding film surrounding a bottom surface of the hot pack; a first integral shielding film surrounding the front, bottom and rear surfaces of the hot pack; and a second integral shielding film surrounding the front, top, and rear surfaces of the hot pack.

In accordance with yet another aspect of the present invention, a continuous thermotherapy device includes: a hot pack; and a hot pack pouch receiving the hot pack and formed of a flexible material to enclose a bent portion.

The hot pack may include: a pyrogen; and a housing receiving the pyrogen therein and having an internal space larger than a volume of the pyrogen such that the pyrogen is mixed within the internal space.

The housing may include: a front plate; a rear plate having the same shape as the front plate and stitched at upper and lower ends thereof to upper and lower ends of the front plate, respectively; a right plate connected at a front end thereof to the front plate and at a rear end thereof to the rear plate; and a left plate having the same shape as the right plate and facing the right plate, the left plate being connected at a front end thereof to the front plate and at a rear end thereof to the rear plate, wherein a width of each of upper and lower ends of the left and right plates may be smaller than a width of a central portion of each of the left and right plates.

The continuous thermotherapy device may further include: an outer hot pack pouch receiving the hot pack pouch therein such that an amount of air contacting the hot pack is adjusted.

The continuous thermotherapy device may further include: an air shielding film surrounding the hot pack such that an amount of air contacting the hot pack is adjusted, wherein the air shielding film may include at least one of a front shielding film surrounding a front surface of the hot pack; a rear shielding film surrounding a rear surface of the hot pack; a top shielding film surrounding a top surface of the hot pack; a bottom shielding film surrounding a bottom surface of the hot pack; a first integral shielding film surrounding the front, bottom and rear surfaces of the hot pack; and a second integral shielding film surrounding the front, top, and rear surfaces of the hot pack.

In accordance with yet another aspect of the present invention, a continuous thermotherapy device includes: a belt; an inner receiving box disposed inside the belt; and a hot pack inserted into the inner receiving box, wherein the hot pack may include: a pyrogen; and a housing receiving the pyrogen therein and having an internal space larger than a volume of the pyrogen such that the pyrogen is mixed within the internal space.

The housing may include: a front plate; a rear plate having the same shape as the front plate and stitched at upper and lower ends thereof to upper and lower ends of the front plate, respectively; a right plate connected at a front end thereof to the front plate and at a rear end thereof to the rear plate; and a left plate having the same shape as the right plate and facing the right plate, the left plate being connected at a front end thereof to the front plate and at a rear end thereof to the rear plate, wherein a width of each of upper and lower ends of the left and right plates may be smaller than a width of a central portion of each of the left and right plates, and wherein the belt may be formed at one end thereof with a female Velcro tape and at the other end thereof with a male Velcro tape, and the opposite ends of the belt may be attached to or detached from each other through attachment/detachment of the female and male Velcro tapes.

The belt may be provided at one end thereof with a plurality of female Velcro tapes separated from each other, the belt may form a ring shape by attaching the male Velcro tape to the female Velcro tapes, and a peripheral length of the ring shape formed by the belt may vary depending on a location of the female Velcro tape to which the male Velcro tape is attached.

According to the present invention, an amount of air contacting a hot pack is adjusted such that the temperature of the hot pack suitable for human bodies can be stably maintained for a long duration, thereby enhancing usability of the continuous thermotherapy device.

### BRIEF DESCRIPTION OF DRAWINGS

FIG. 1 is a perspective view of a continuous thermotherapy device according to a first embodiment of the present invention;

FIG. 2 is a perspective view of the continuous thermotherapy device according to the first embodiment of the present invention when viewed from an opposite side to FIG. 1;

FIG. 3 shows the continuous thermotherapy device according to the first embodiment of the present invention, in which hot pack cases are received in a receiving box;

FIG. 4 shows the continuous thermotherapy device according to the first embodiment of the present invention, in which opposite ends of a belt are coupled to each other;

FIG. 5 is a perspective view of a hot pack case of the continuous thermotherapy device according to the first embodiment of the present invention;

FIG. 6 is a perspective view of a hot pack of the continuous thermotherapy device according to the first embodiment of the present invention;

FIG. 7 shows a process in which the hot pack of the continuous thermotherapy device according to the first embodiment of the present invention is folded;

FIG. 8 shows a state in which the hot pack of the continuous thermotherapy device according to the first embodiment of the present invention is folded;

FIG. 9 shows a coupling relation between the hot pack, the hot pack case, and the receiving box of the continuous thermotherapy device according to the first embodiment of the present invention;

FIG. 10 is a perspective view of the hot pack without an air shielding film in the continuous thermotherapy device according to the first embodiment of the present invention;

FIG. 11 shows a state in which a front shielding film encloses a front surface of the hot pack in the continuous thermotherapy device according to the first embodiment of the present invention;

FIG. 12 shows a state in which front and rear shielding films enclose front and rear surfaces of the hot pack, respectively, in the continuous thermotherapy device according to the first embodiment of the present invention;

FIG. 13 shows a state in which front, top, and rear shielding films enclose front, top, and rear surfaces of the hot pack, respectively, in the continuous thermotherapy device according to the first embodiment of the present invention;

FIG. 14 shows a state in which the front, top, rear, and bottom shielding films enclose front, top, rear, and bottom surfaces of the hot pack, respectively, in the continuous thermotherapy device according to the first embodiment of the present invention;

FIG. 15 shows a state in which a first integral shielding film encloses the front, bottom and rear surfaces of the hot pack in the continuous thermotherapy device according to the first embodiment of the present invention;

FIG. 16 shows a state in which the first integral shielding film encloses the front, bottom and rear surfaces of the hot pack and the top shielding film encloses the top surface of the hot pack in the continuous thermotherapy device according to the first embodiment of the present invention;

FIG. 17 shows a state in which the first integral shielding film encloses the front, bottom and rear surfaces of the hot pack and a second integral shielding film encloses the front and rear surfaces and the top surface of the hot pack in the continuous thermotherapy device according to the first embodiment of the present invention;

FIG. 18 shows a state in which the hot pack case receiving the hot pack is inserted into the receiving box in the continuous thermotherapy device according to the first embodiment of the present invention;

FIG. 19 shows a state in which the hot pack expands in the continuous thermotherapy device according to the first embodiment of the present invention;

FIG. 20 is a perspective view of a hot pack pouch of a continuous thermotherapy device according to a second embodiment of the present invention;

FIG. 21 shows a state in which a hot pack is inserted into the hot pack pouch in the continuous thermotherapy device according to the second embodiment of the present invention;

FIG. 22 shows a state in which the hot pack is received in the hot pack pouch in the continuous thermotherapy device according to the second embodiment of the present invention;

FIG. 23 shows a state in which the hot pack is enclosed by a plurality of hot pack pouches in the continuous thermotherapy device according to the second embodiment of the present invention;

FIG. 24 shows a state in which the continuous thermotherapy device according to the first and second embodiments of the present invention is received in a vest;

FIG. 25 is a plan view of a continuous thermotherapy device according to a first modified embodiment of the present invention;

FIG. 26 is a bottom view of the continuous thermotherapy device according to the first modified embodiment of the present invention;

FIG. 27 is a first perspective view of the continuous thermotherapy device according to the first modified embodiment of the present invention;

FIG. 28 is a second perspective view of the continuous thermotherapy device according to the first modified embodiment of the present invention;

FIG. 29 is a plan view of a continuous thermotherapy device according to a second modified embodiment of the present invention;

FIG. 30 is a bottom view of the continuous thermotherapy device according to the second modified embodiment of the present invention;

FIG. 31 is a first perspective view of the continuous thermotherapy device according to the second modified embodiment of the present invention;

FIG. 32 is a second perspective view of the continuous thermotherapy device according to the second modified embodiment of the present invention;

FIG. 33 is a graph showing temperature change of a hot pack in a case where the hot pack is left in the air;

FIG. 34 is a graph showing temperature change of a hot pack in a case where the hot pack is received in a hot pack case; and

FIG. 35 is a graph showing temperature change of a hot pack in a case where the hot pack is received in a hot pack case while being enclosed by an air shielding film.

### DETAILED DESCRIPTION

Hereinafter, exemplary embodiments of the present invention will be described with reference to the accompanying drawings.

It should be noted that the drawings are not to precise scale and may be exaggerated in thickness of lines or size of components for descriptive convenience and clarity only.

Furthermore, terms used herein are defined by taking functions of the present disclosure into account and can be changed according to the custom or intention of users or operators.

Therefore, definition of the terms should be made according to the overall disclosure set forth herein.

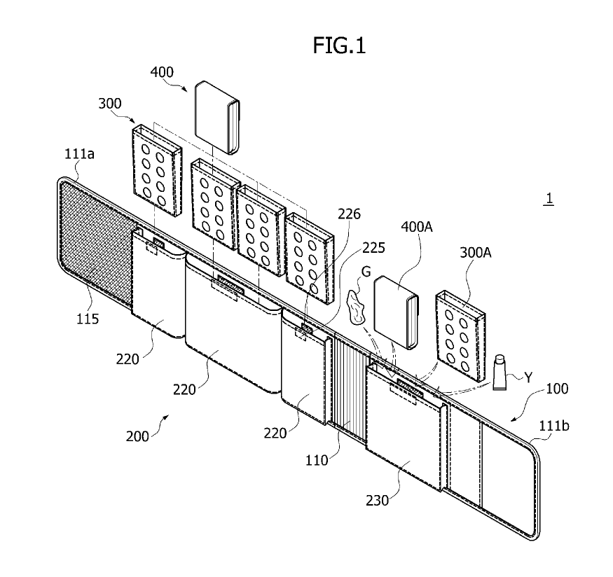

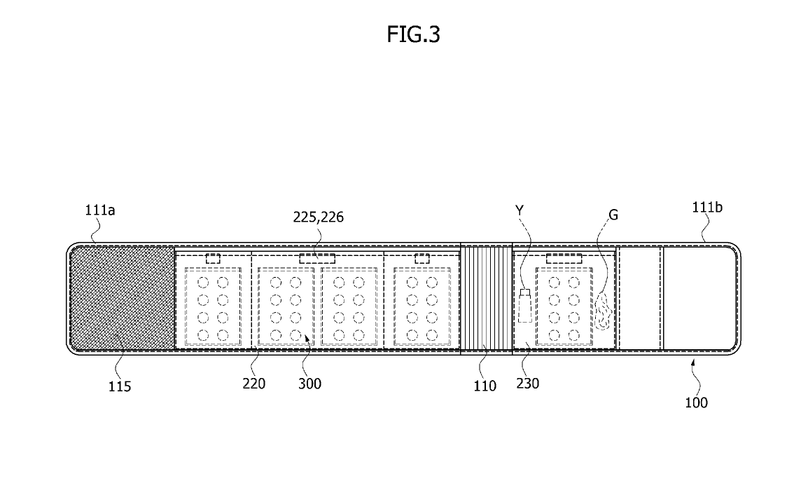

Referring to FIGS. 1 to 4, a continuous thermotherapy device 1 according to a first embodiment of the present invention includes a belt 100, a receiving box 200, hot pack cases 300, hot packs 400, and air shielding films 500.

The belt 100 is formed of at least one of nylon, spandex, and gore-tex.

Nylon is a heat resistant material. Accordingly, when the belt 100 is formed of the nylon, the belt 100 can resist deformation by heat transferred from the hot packs 400 or the hot pack cases 300 which receive the hot packs 400.

Spandex is a common name of synthetic fibers formed of elastic fibers of polyurethane and has excellent flexibility. Accordingly, when the belt 100 is worn on the human abdomen (hereinafter, referred to as “wearing position”), wearing comfort of the belt 100 can be improved by flexibility of the spandex.

Gore-tex refers to a very thin film prepared by a process in which a Teflon-based resin having excellent heat resistance and chemical resistance is stretched and heated and numerous small holes are punched therethrough. When the belt 100 is formed of gore-tex, although external moisture such as rainwater does not permeate into the belt 100, sweat generated within the belt 100 according to an operation of the continuous thermotherapy device 1 is discharged to the outside of the belt 100, thereby solving a problem of hot moisture frequently occurring in waterproof products.

The belt 100 has a length and a shape capable of surrounding a human body part, for example, the abdomen. According to the present embodiment, while the belt 100 is formed in a shape of an abdominal binder having a much greater length in a left-right direction than that in an up-down direction, the belt 100 may be replaced by any other form capable of receiving the hot packs 400 or the hot pack cases 300.

In this embodiment, the belt 100 is formed at one end 111a thereof with a female Velcro tape 115 and at the other end 111b thereof with a male Velcro tape 116.

The opposite ends 111a and 111b of the belt 100 are detachably coupled to each other through attachment/detachment of the female and male Velcro tapes 115 and 116.

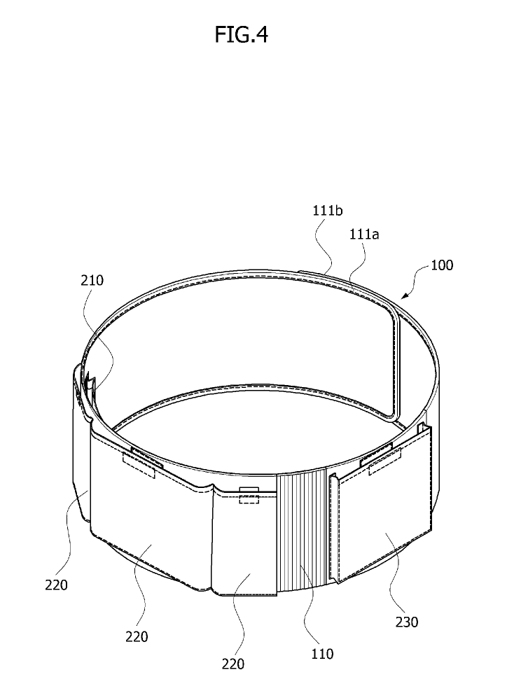

FIG. 4 shows a state in which the opposite ends 111a and 11b of the belt 100 are coupled to each other.

The belt 100 is provided with a flexible portion 110. The flexible portion 110 is formed of a flexible material. Accordingly, the belt 100 may be easily worn on a wearing position due to flexibility of the flexible portion 110, and when the belt 100 is completely worn, an inner side of the belt 100 may be attached to the wearing position by a restoring force of the flexible portion 110. An improved thermal effect through the continuous thermotherapy device 1 can be achieved according to improvement of attaching force.

Although one flexible portion 100 is provided to the belt 100 in the present embodiment, at least two flexible portions may be provided to a plurality of points of the belt 100, without being limited thereto.

The receiving box 200 is provided to the belt 100. The receiving box 200 includes inner receiving boxes 210 disposed inside the belt 100, outer receiving boxes 220 disposed outside the belt 100, and an auxiliary receiving box 230 disposed inside or outside the belt 100.

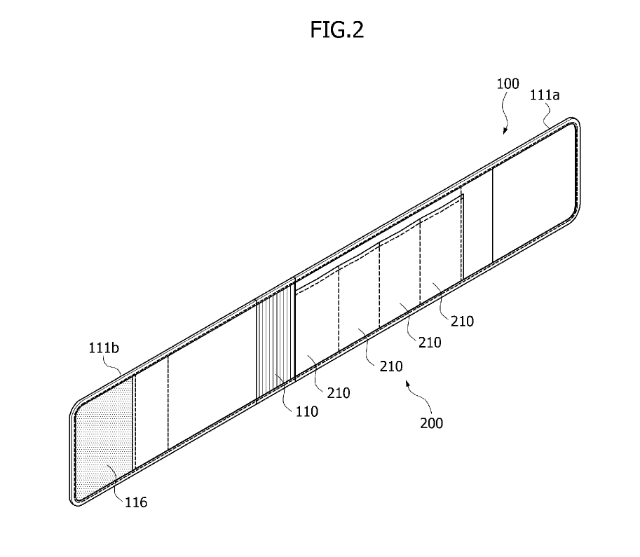

The inner receiving boxes 210 (shown in FIG. 2) are open at upper sides thereof and the hot packs 400 or the hot pack cases 300 are inserted into the inner receiving boxes 210 through the open upper sides, respectively.

The outer receiving boxes 220 (shown in FIG. 1) are located to face the inner receiving boxes 220, with the belt 100 interposed therebetween. The outer receiving boxes 220 also are open at upper sides thereof and the hot packs 400 or the hot pack cases 300 are inserted into the outer receiving boxes 220 through the open upper sides, respectively.

Although both the inner and outer receiving boxes 210, 220 are formed in the belt 100 in the present embodiment, any one of the inner and outer receiving boxes 210, 220 may also be formed in the belt 100, without being limited thererto.

A female Velcro tape 225 is provided to an inner surface of an upper end of each of the outer receiving boxes 220, and a male Velcro tape 226 is formed on an outer surface of the belt 100 facing the inner surface of the upper end of the outer receiving box 220.

The female and male Velcro tapes 225, 226 are disposed to face each other. The upper end of the outer receiving box 220 is detachably coupled to the belt 100 through attachment/detachment of the female and male Velcro tapes 225, 226.

Accordingly, the hot pack 400 and the hot pack case 300 received in the outer receiving box 220 are prevented from being unintentionally separated from the outer receiving box 220.

Although the Velcro tapes 225, 226 are formed on the outer receiving box 220 and on the outer surface of the belt 100, respectively, in the present embodiment, the Velcro tapes may also be formed on the inner receiving boxes 210 or the auxiliary receiving box 230 such that an upper end thereof is closed, without being limited thereto.

The auxiliary receiving box 230 (shown in FIG. 1) is open at an upper side thereof, and receives a spare hot pack 400A, a spare hot pack case 300A, a burn ointment Y, a plastic bag G for preserving the continuous thermotherapy device when not in use, a disposable band-aid, or the like through the open upper sides thereof.

Although the auxiliary receiving box 230 is formed outside the belt 100 in the present embodiment, the auxiliary receiving box 230 may also be formed inside the belt 100.

An upper end of the belt 100 is located higher than those of the inner and outer receiving boxes 210, 220. Accordingly, the hot pack cases 300 into which the hot packs 400 are inserted are received in the inner receiving boxes 210 or the outer receiving boxes 220, respectively, and then the upper end of the belt 100 is folded twoard the open upper sides of the inner or outer receiving boxes 210 or 220, thereby preventing heat dissipation through the open upper sides of the inner or outer receiving boxes 210 or 220.

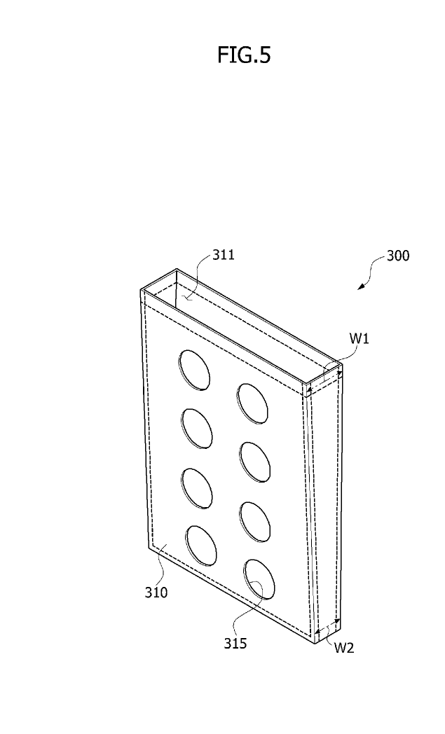

Referring to FIGS. 1 to 5, each of the hot pack cases 300 includes an opening 311 formed at an upper side thereof and a case main body 310 having a hexahedral shape.

Since the hexahedral shape of the case main body 310 results from a shape of the hot pack 400, the three dimensional shape of the case main body 310 may be changed corresponding to the shape of the hot pack 400 when the shape of the hot pack 400 is replaced by another shape.

The case main body 310 is formed such that an upper thickness W1 of a side surface is greater than a lower thickness W2 of the side surface. Accordingly, even if gravity is applied to the hot pack 400 for a long period of time as the hot pack 400 is continuously received in the hot pack case 300, the hot pack case 300 can prevent a pyrogen 410 of the hot pack 400 from leaning to a direction of gravity, namely, a downward direction.

If the pyrogen 410 leans to a specific portion of the hot pack 400, a large amount of heat is radiated from the specific portion of the hot pack 400 such that a user may have a burn when the user body contact the heat for a long period of time. However, the burn can be easily prevented due to the shape of the case main body 310 in which the thickness of the side surface is decreased from top to bottom.

The hot pack case 300 encloses the hot pack 400 and is received in the receiving box 200. A plurality of air contact holes 315 is formed on front and rear surfaces of the hot pack case 300. An amount of external air contacting the hot pack 400 received in the hot pack case 300 may be increased by the air contact holes 315.

Since the hot pack case 300 directly receives heat from the hot pack 400 received therein, the hot pack case 300 should be formed of a material having heat resistance. According to the present embodiment, the hot pack case 300 is formed of nylon.

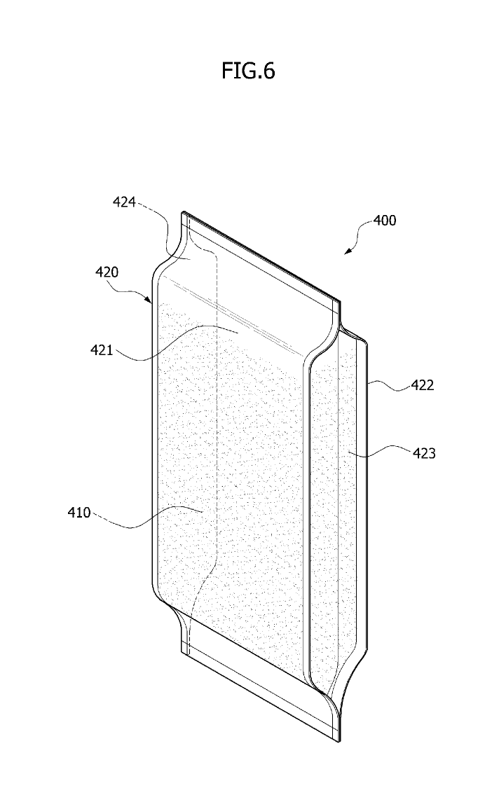

Referring to FIGS. 6 to 8, each of the hot packs 400 includes a pyrogen 410 and a housing 420 accommodating the pyrogen 410 therein.

The exothermic principle of the hot pack 400 is to use chemical energy generated in oxidation reaction in which an iron component is changed to iron oxide. The pyrogen 410 includes aluminum, magnesium, and the like, acting as a catalyst, in addition to the iron component.

Further, the pyrogen 410 includes diatomite, a synthetic resin, and the like for preventing exothermic reaction from being terminated in an instant due to simultaneous chemical actions of reactants. A rate at which reactants within the pyrogen 410 combine to cause exothermic reaction is adjusted through the diatomite, the synthetic resin, and the like, and the amount of radiated heat is adjusted by an amount of air contact.

The housing 420 has an internal space greater than a total volume of the pyrogen 410 such that the reactants of the pyrogen 410 may be sufficiently mixed in the housing 420. The reason why the internal space is formed to be sufficiently large relative to the pyrogen 410 is to break the pyrogen 410 into small pieces and mix them again in the internal space when the pyrogen 410 is conglomerated according to progress of exothermic reaction.

The housing 420 has a shape, as shown in FIG. 6. The housing 420 is formed such that a front plate 421 and a rear plate 422 may be folded.

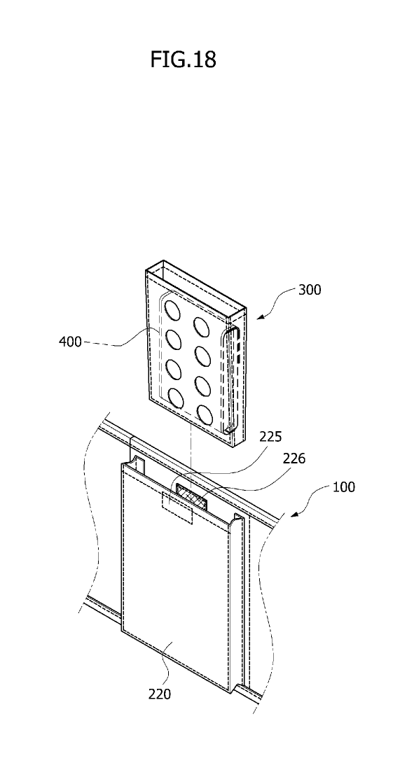

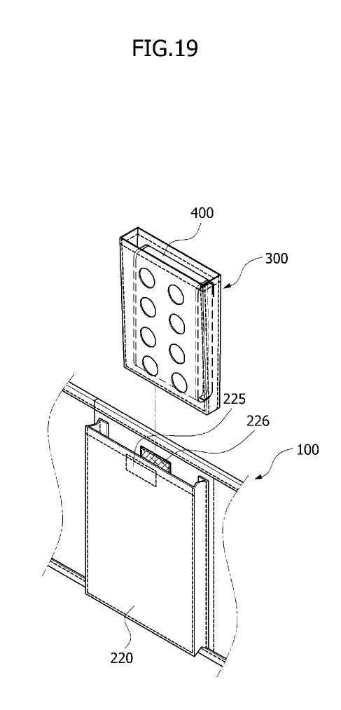

The pyrogen 410 expands over time after exothermic reaction is initiated, in which case the folded front and rear plates 421, 422 buffer expansion of the pyrogen 410 while being unfolded according to the degree of expansion (see FIGS. 18 and 19).

In the related art, the housing is formed in a shape of a simple envelope and thus, when the pyrogen expands, only the expanded portion convexly thickens, in which the convex portion is further attached to skin and other portions are separated from the skin, such that the skin contacting the convex portion is burned.

However, according to the present invention, even if the pyrogen 410 expands, the foldable front and rear plates 421, 422 sufficiently buffer the expansion to prevent only the expanded portion from being convexly thickened, thereby restricting a local burn.

The housing 420 includes the front plate 421, the rear plate 422, a right plate 423, and a left plate 424.

The front plate 421 configures a front surface of the housing 420 and is formed in a rectangular shape. The rear plate 422 configures a rear surface of the housing 420 and is formed in a rectangular shape identically to the front plate 421. Upper and lower ends of the rear plate 422 are stitched to upper and lower ends of the front plate 421, respectively.

The right plate 423 configures a right surface (based on FIG. 6) of the housing 420. A front end of the right plate 423 is connected to the front plate 421 and a rear end of the right plate 423 is connected to the rear plate 422. The right plate 423 is formed such that a width of each of upper and lower ends is less than that of a central portion (see FIG. 6).

The left plate 424 faces the right plate 423 and configures a left surface of the housing 420. A front end of the left plate 424 is connected to the front plate 421 and a rear end of the left plate 424 is connected to the rear plate 422. The left plate 424 is formed such that a width of each of upper and lower ends is less than that of a central portion (see FIG. 6).

Since the internal space of the housing 420 is larger than the volume of the pyrogen 410, compact insertion of the hot pack 400 into the hot pack case 300 is realized by folding the housing 420.

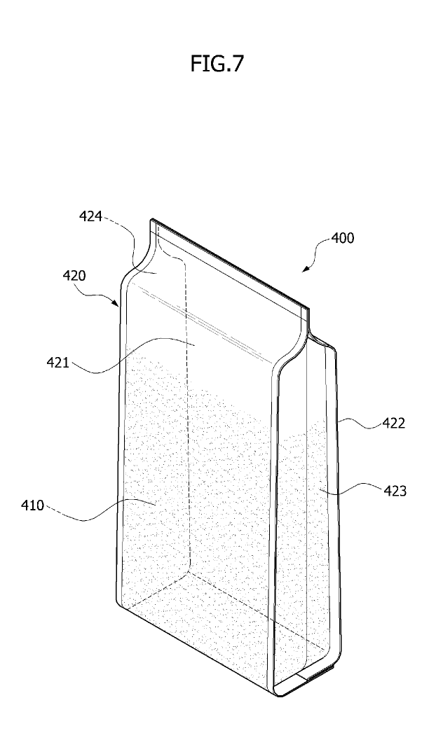

For folding the housing 420, a lower portion of each of the front and rear plates 421, 422 is first folded as shown in FIG. 7. In this case, a width of a lower end of the left and right plates 424, 423 is made identical to that of the central portion thereof. Accordingly, front and rear widths of the pyrogen 410 embedded in a lower portion of the housing 420 are made identical to those of the pyrogen 410 embedded in a central portion of the housing 420.

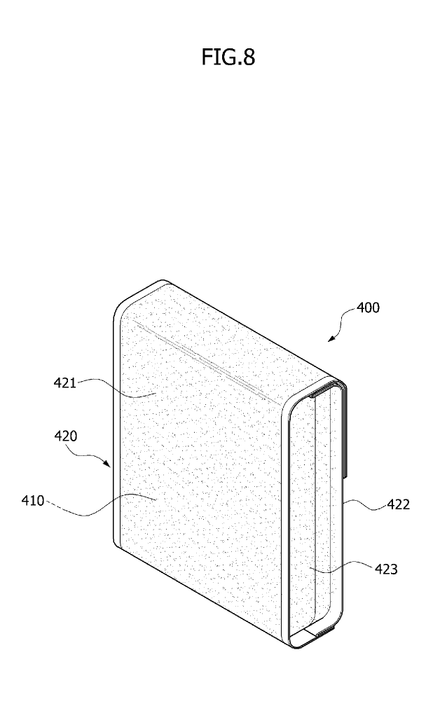

Next, an upper portion of each of the front and rear plates 421, 422 is folded as shown in FIG. 8. In this case, a width of an upper end of each of the left and right plates 424, 423 is made identical to the width of the central portion thereof. Accordingly, front and rear widths of the pyrogen 410 embedded in an upper end of the housing 420 are made identical to those of the pyrogen 410 embedded in the central portion of the housing 420.

The hot pack 400 is inserted in the shape shown in FIG. 8 into the hot pack case 300 (see FIG. 18). Since the hot pack 400 is compactly inserted into the hot pack case 300, an internal space of the hot pack case 300 has the same volume as that of the hot pack 400 folded as shown in FIG. 8.

The housing 420 may be unfolded or folded, as described above. With the housing unfolded, the pyrogen 410 accommodated in the housing 420 may be easily mixed and the conglomerated pyrogen 410 may also be broken into small pieces. Further, when the hot pack 400 is inserted into the hot pack case 300, an outer shape of the hot pack 400 can be changed to exactly fit the hot pack case 300 through folding of the housing 420.

Referring to FIGS. 6 to 8, the pyrogen 410 of the hot pack 400 is not conglomerated over time, if temperature is adjusted only by interrupting air, while the hot pack 400 is not inserted into the hot pack case 300 and is maintained in a state of having a large empty space therein. Accordingly, when shaken, the hot pack 400 feels as though there is sand therein, while making a crunching sound.

It can be seen that lifetime of the hot pack 400 in this state is made a little longer at a temperature lower than that of the hot pack left in air without interruption of air. The hot pack 400 in this state is further heated up and is more rapidly expired, as compared with the case of compactly filling the hot pack case 300 with the hot pack 400. The hot pack 400 in this state cools within about 24 hours to about 36 hours after exothermic reaction is initiated. In the case of compactly filling the hot pack case 300 with the hot pack 400, the hot pack 400 is maintained at a temperature of 40° C. or higher for 48 hours to 60 hours.

The reason for such difference is because vapor generated during exothermic reaction is not discharged to the outside and does not dry while the pyrogen 410 does not move within the housing 420, when exothermic reaction progresses in a state in which there is no empty space within the hot pack 400, namely, the hot pack 400 is compactly received within the hot pack case 300. The pyrogen 410 is conglomerated to have a size as small as a bean when a period of time ranging from 6 hours to 8 hours passes after the reaction is initiated. The pyrogen 410 may harden in a shape similar to soft rice cake dough according to compositions thereof, and there is a harder portion like a bean within the dough.

When the conglomerated pyrogen 410 is lightly pressed or rubbed through pressing of the housing 420 while the pyrogen 410 is conglomerated, the hard portion of the pyrogen 410 is broken and, at the same time, the pyrogen 410 is changed in the original shape similar to sand. Accordingly, the temperature of the hot pack 400 is also increased while exothermic reaction is more actively triggered.

Although the pyrogen 410 returns to the shape similar to sand through breaking of the conglomerated portion thereof, the pyrogen 410 is conglomerated again after 6 to 8 hours. If the conglomerated portion of the pyrogen 410 is broken again and arranged in that case, exothermic reaction is activated in the pyrogen 410.

If the pyrogen 410 is repeatedly conglomerated and broken, a particle size of the broken pyrogen 410 is gradually increased, and if such a process is repeated six to nine times, the hot pack 400 is maintained at a predetermined temperature and lastly, is not further heated.

If the pyrogen 410 is conglomerated within the housing by compactly filling the hot pack case 300 with the hot pack 400, while air contact may occur and exothermic reaction may be triggered on a surface of the conglomerated pyrogen 410, the reaction within the conglomerated pyrogen 410 is stopped. Accordingly, if the pyrogen 410 is not artificially broken and mixed again, the reaction rate of the pyrogen 410 decreases and desired temperature is not achieved.

As described above, temperature adjustment and lifetime extension of the hot pack 400 can be achieved through repetition of conglomeration (see FIG. 8) and dispersion (see FIG. 6), and thus, an advantage of the present invention is that the housing 420 is formed in a shape of a rice bag such that conglomeration and dispersion can be easily performed.

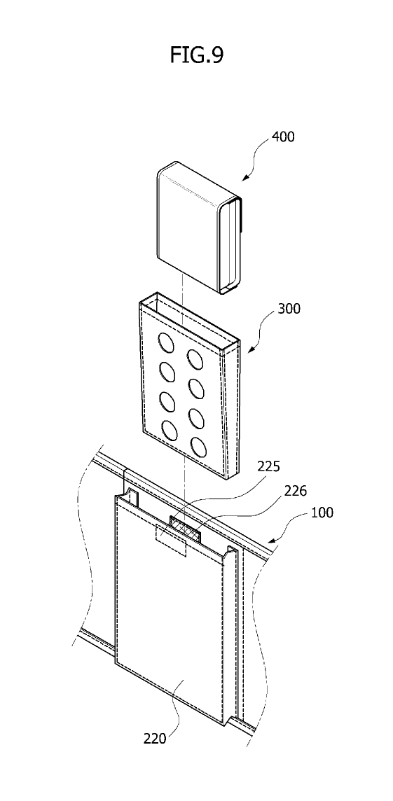

Conglomeration and dispersion processes include a process (see FIG. 9) in which the pyrogen 410 is gathered at a lower side of an internal space of the housing 420 through folding the housing 420 and the hot pack case 300 including the hot pack 400 therein is placed in the outer receiving box 220, a process (see FIG. 6) in which the hot pack 400 is withdrawn from the hot pack case 300, broken into small pieces, and evenly mixed, when exothermic reaction is initiated such that the pyrogen 410 is conglomerated, a process (see FIG. 7) in which the pyrogen 410 broken into the small pieces similar to sand is gathered in the lower side of the internal space of the housing 420, and a process (see FIG. 9) in which the hot pack 400 is placed in the hot pack case 300 and the hot pack case 300 is placed in the outer receiving box 200 again.

With advancing years, a person has a decreased quantity of motion and is less exposed to a work lifting heavy things or requiring a large amount of energy, so that strength of a hand and an arm is sapped. Accordingly, gripping the hot pack 400 several times a day can increase strength of fingers and prevent dementia.

Here, the hot pack 400 of the present invention is a term including a number of thermal products, which can cause exothermic reaction, such as a hot poultice, a hand warmer, an electrical pack, a fomentation pack, a massage pack, and the like in addition to products referred to as a hot pack.

Each of the air shielding films 500 encloses the hot pack 400 to prevent contact between the hot pack 400 and air. Temperature of the hot pack 400 may be adjusted through the air shielding film 500. Namely, although the temperature of the hot pack 400 may not be directly adjusted, transfer of heat from the hot pack 400 to the outside may be adjusted through the air shielding film 500 surrounding the hot pack 400.

The air shielding film 500 includes a front shielding film 510, a rear shielding film 520, a top shielding film 530, a bottom shielding film 540, a first integral shielding film 550, and a second integral shielding film 560, and each of the shielding films may be used alone or a plurality of shielding films may be simultaneously used.

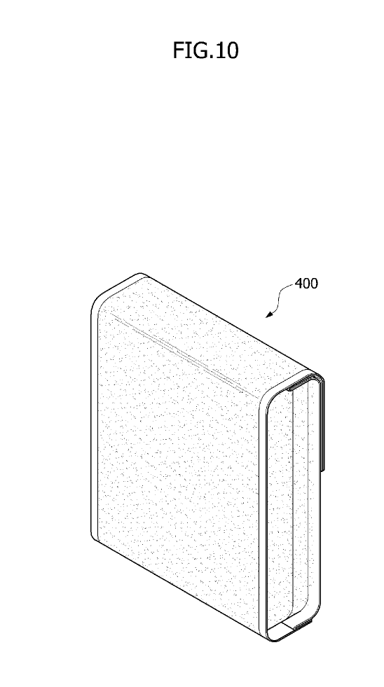

FIG. 10 shows a hot pack 400 that is not enclosed by the air shielding film 500. When temperature of the hot pack 400 is low, only the hot pack 400 is inserted into the hot pack case 300 without using the air shielding film 500.

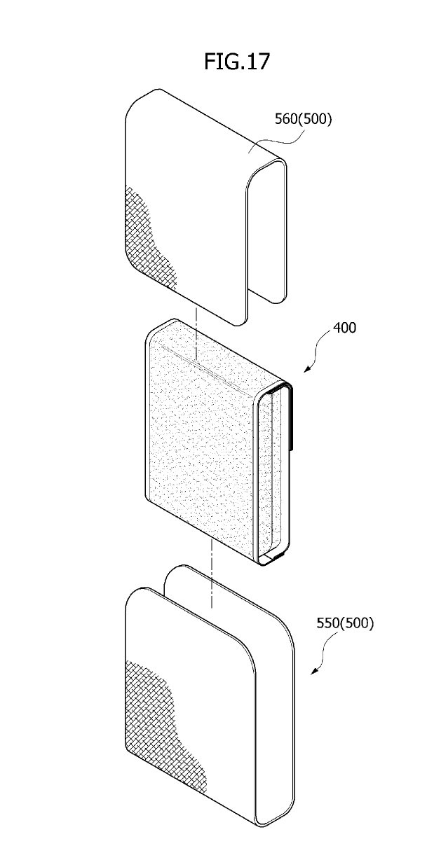

FIG. 17 shows a state in which the first integral shielding film 550 encloses front, bottom and rear surfaces of the hot pack 400 and the second integral shielding film 560 encloses the front, top, and rear surfaces of the hot pack 400. This is a state in which the highest shielding rate is achieved through the air shielding film 500, and the first and second integral shielding films 550, 560 are used when the hot pack 400 has the highest temperature.

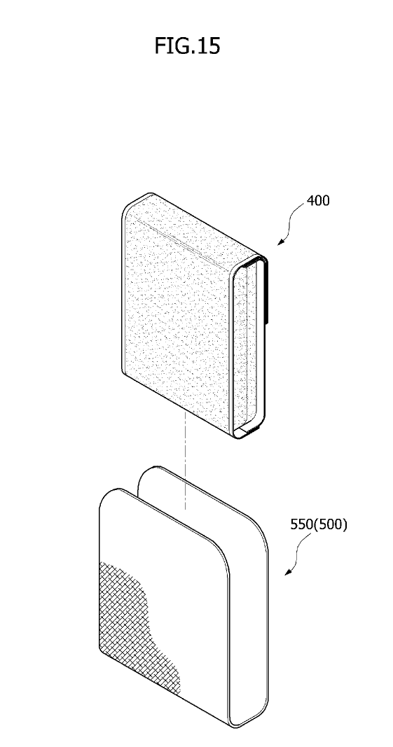

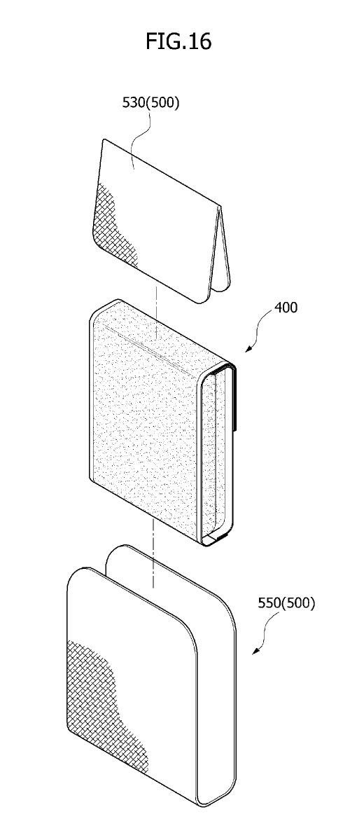

FIG. 16 shows a state in which the first integral shielding film 550 encloses the front, bottom and rear surfaces of the hot pack 400 and the top shielding film 530 encloses the top surface of the hot pack 400. In this case, when the temperature of the hot pack 400 is decreased, the top shielding film 530 is removed and thus, a state in which the first integral shielding film 550 encloses the front, bottom and rear surfaces of the hot pack 400 can be easily realized as shown in FIG. 15.

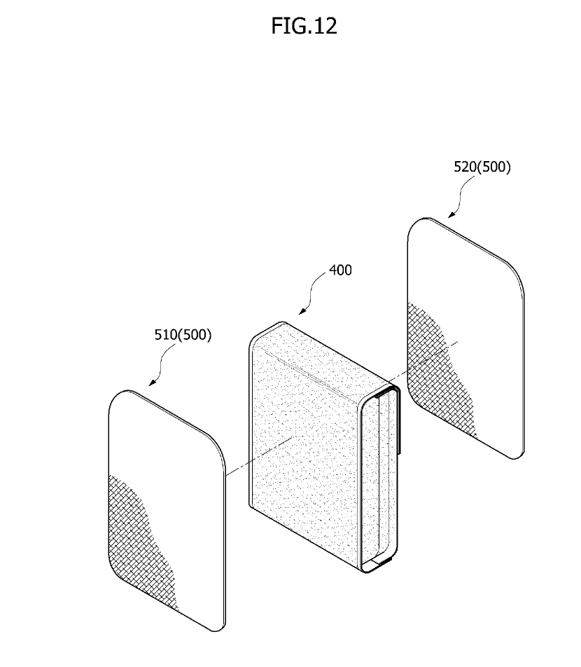

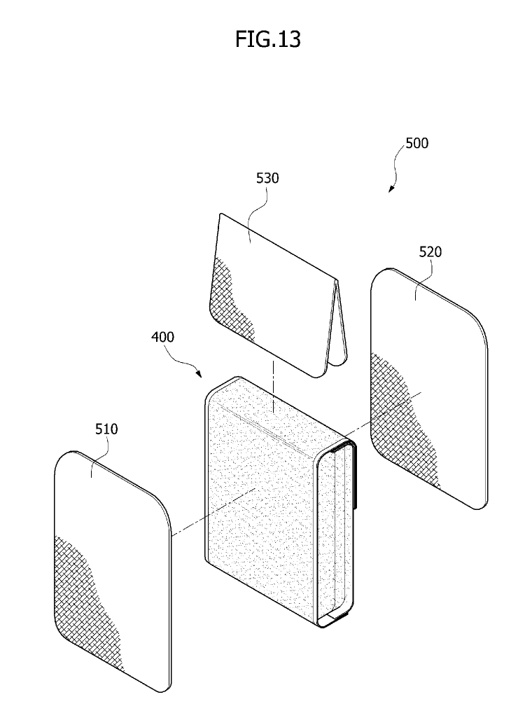

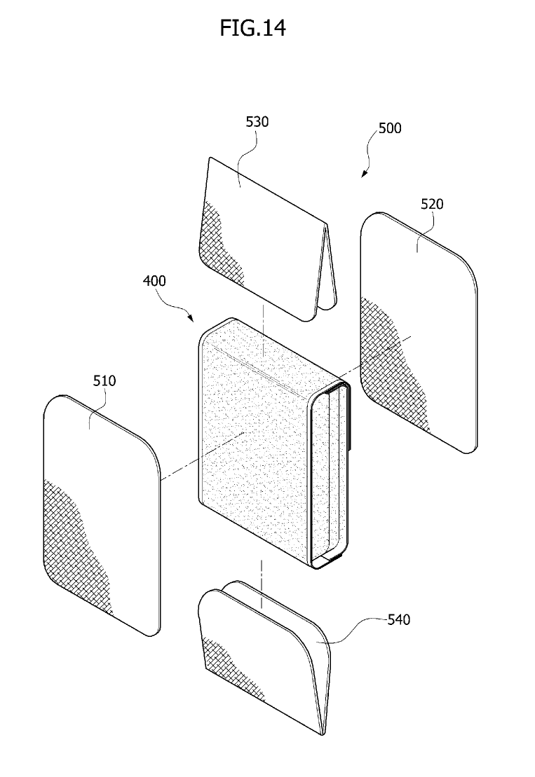

FIG. 14 shows a state in which the front shielding film 510 encloses the front surface of the hot pack 400, the rear shielding film 520 encloses the rear surface of the hot pack 400, the top shielding film 530 encloses the top surface of the hot pack 400, and the bottom shielding film 540 encloses the bottom surface of the hot pack 400. When the bottom shielding film 540 is removed in the state as shown in FIG. 14, a state as shown in FIG. 13 is realized, and when the top and rear shielding films 530, 520 are sequentially removed, states as shown in FIGS. 12 and 13 are realized in sequence. Further, when the front shielding film 510 is removed, a state in which the air shielding film 500 is omitted is realized as shown in FIG. 10.

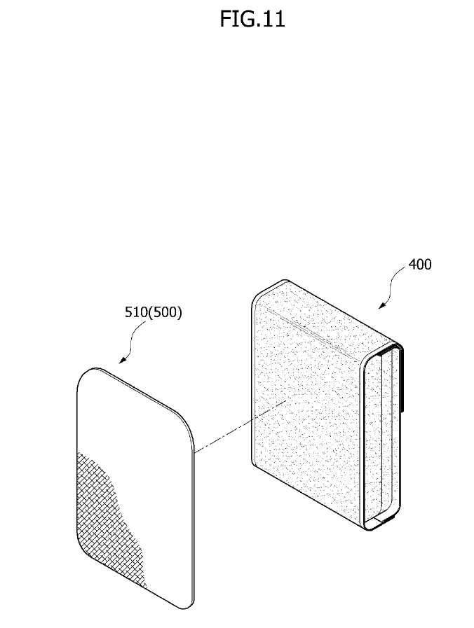

Since temperature of the hot pack 400 suitable for thermotherapy ranges from 40° C. to 45° C., the hot pack 400 is enclosed by the air shielding film 500 to reach the desired temperature through the methods as shown in FIGS. 11 to 17. When the air shielding film 500 is omitted and only the hot pack 400 is received in the hot pack case 300, the hot pack case 300 is first received in the outer receiving box 220 and then is moved to the inner receiving box 210 after a predetermined period of time, according to the temperature of the hot pack 400.

Hereinafter, a continuous thermotherapy device according to a second embodiment of the invention will be described. Since the continuous thermotherapy device according to the second embodiment is the same as the continuous thermotherapy device according to the first embodiment except that the hot pack case 300 is replaced by a hot pack pouch 350, descriptions of the same components as those of the continuous thermotherapy device according to the first embodiment will be omitted.

Referring to FIGS. 1 to 4, the continuous thermotherapy device according to the second embodiment of the present invention includes a belt 100, a receiving box 200, hot pack pouches 350, hot packs 400, and air shielding films 500.

The belt 100 is formed of at least one of nylon, spandex, and gore-tex. In this embodiment, the belt 100 is formed at one end 111a thereof with a female Velcro tape 115 and at the other end 111b thereof with a male Velcro tape 116.

The belt 100 is provided with a flexible portion 110. The flexible portion 110 is formed of a flexible material. Although one flexible portion 100 is included in the belt 100 according to the present embodiment, at least two flexible portions may be provided at a plurality of points of the belt 100, without being limited thereto.

The receiving box 200 is provided to the belt 100. The receiving box 200 includes inner receiving boxes 210 disposed inside the belt 100, outer receiving boxes 220 disposed outside the belt 100, and an auxiliary receiving box 230 disposed inside or outside the belt 100.

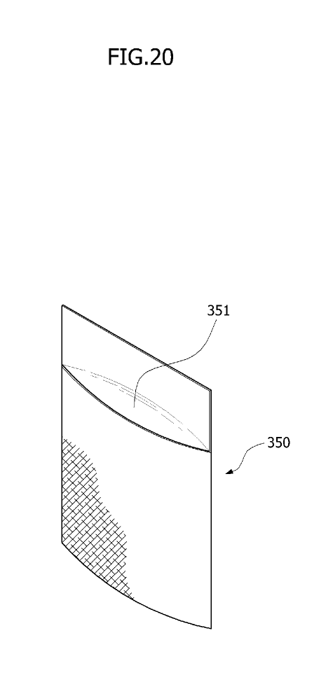

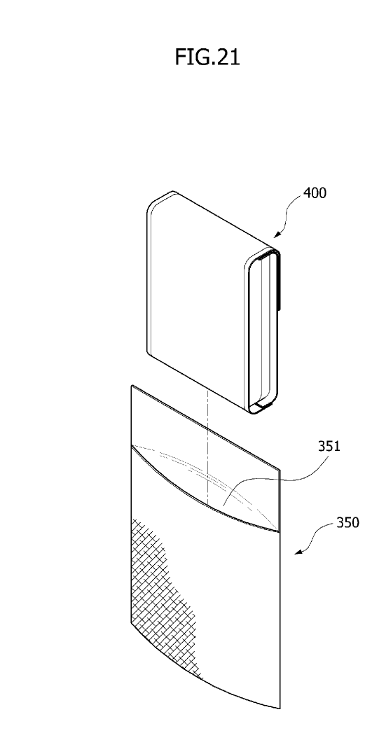

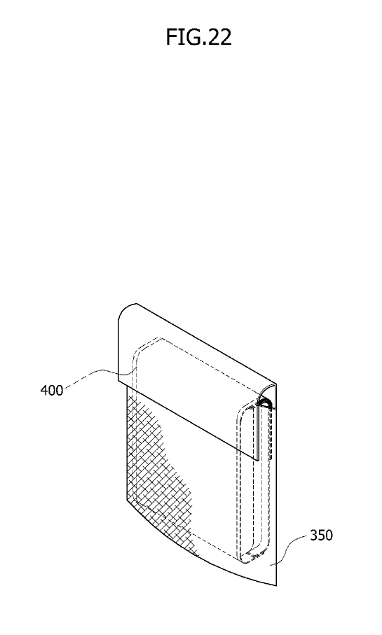

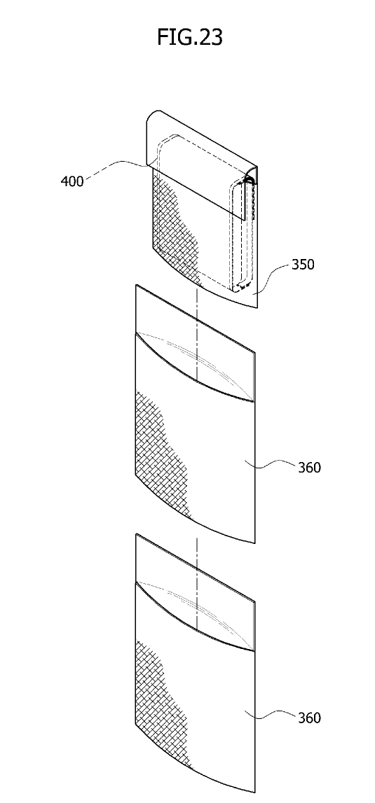

Referring to FIGS. 20 to 23, each of the hot pack pouches 350 has an opening 351 formed at an upper portion thereof and is formed in a shape of a document envelope. The hot pack pouch 350 is formed of a flexible material to enclose a bent portion such as a knee, an elbow, and the like.

In this embodiment, the hot pack pouch 350 is formed of non-woven fabrics. An amount of air contacting the hot pack 400 may be adjusted through the hot pack pouch 350 surrounding the hot pack 400 and an outer hot pack pouch 360 in addition to the air shielding film 500. A hot pack pouch additionally surrounding the hot pack 400 is referred to as the outer hot pack pouch 360, and the outer hot pack pouch 360 may also be formed of non-woven fabrics.

Referring to FIGS. 6 to 8, each of the hot packs 400 includes a pyrogen 410 and a housing 420 accommodating the pyrogen 410.

The housing 420 has a shape as shown in FIG. 6. The housing 420 is formed such that a front plate 421 and a rear plate 422 may be folded.

The pyrogen 410 expands over time after exothermic reaction is initiated, in which case the folded front and rear plates 421, 422 buffer expansion of the pyrogen 410 while being unfolded according to the degree of expansion (see FIGS. 18 and 19).

However, according to the present invention, even if the pyrogen 410 expands, the foldable front and rear plates 421, 422 sufficiently buffer the expansion to prevent only the expanded portion from being convexly thickened, thereby restricting a local burn.

The housing 420 includes the front plate 421, the rear plate 422, a right plate 423, and a left plate 424.

The front plate 421 configures a front surface of the housing 420 and is formed in a rectangular shape. The rear plate 422 configures a rear surface of the housing 420 and is formed in a rectangular shape identically to the front plate 421. Upper and lower ends of the rear plate 422 are stitched to upper and lower ends of the front plate 421, respectively.

The right plate 423 configures a right surface (based on FIG. 6) of the housing 420. A front end of the right plate 423 is connected to the front plate 421 and a rear end of the right plate 423 is connected to the rear plate 422. The right plate 423 is formed such that a width of each of upper and lower ends is less than that of a central portion (see FIG. 6).

The left plate 424 faces the right plate 423 and configures a left surface of the housing 420. A front end of the left plate 424 is connected to the front plate 421 and a rear end of the left plate 424 is connected to the rear plate 422. The left plate 424 is formed such that a width of each of upper and lower ends is less than that of a central portion (see FIG. 6).

Since an internal space of the housing 420 is larger than the volume of the pyrogen 410, compact insertion of the hot pack 400 into the hot pack case 300 is realized by folding the housing 420.

For folding the housing 420, lower portions of the front and rear plates 421, 422 are folded as shown in FIG. 7. In this case, a width of a lower end of each of the left and right plates 424, 423 is made identical to the width of the central portion thereof. Accordingly, front and rear widths of the pyrogen 410 embedded in a lower portion of the housing 420 are made identical to those of the pyrogen 410 embedded in a central portion of the housing 420.

Next, an upper portion of each of the front and rear plates 421, 422 is folded as shown in FIG. 8. In this case, a width of an upper end of each of the left and right plates 424, 423 is made identical to the width of the central portion thereof. Accordingly, front and rear widths of the pyrogen 410 embedded in an upper end of the housing 420 are made identical to those of the pyrogen 410 embedded in the central portion of the housing 420.

The hot pack 400 is inserted in the shape shown in FIG. 8 into the hot pack case 300 (see FIG. 18). Since the hot pack 400 is compactly inserted into the hot pack case 300, an internal space of the hot pack case 300 has the same volume as that of the hot pack 400 folded as shown in FIG. 8.

The housing 420 may be unfolded or folded, as described above. With the housing unfolded, the pyrogen 410 accommodated in the housing 420 may be easily mixed and the conglomerated pyrogen 410 may also be broken into small pieces. Further, when the hot pack 400 is inserted into the hot pack case 300, an outer shape of the hot pack 400 can be changed to exactly fit the hot pack case 300 by folding the housing 420.

The air shielding film 500 encloses the hot pack 400 to prevent contact between the hot pack 400 and air. Temperature of the hot pack 400 may be adjusted through the air shielding film 500. The air shielding film 500 includes a front shielding film 510, a rear shielding film 520, a top shielding film 530, a bottom shielding film 540, a first integral shielding film 550, and a second integral shielding film 560, and each of the shielding films may be used alone and a plurality of shielding films may be simultaneously used.

Since temperature of the hot pack 400 suitable for thermotherapy ranges from 40° C. to 45° C., the hot pack 400 is enclosed by the air shielding film 500 to fit the temperature through the methods, as shown in FIGS. 11 to 17. When the air shielding film 500 is omitted and only the hot pack 400 is received in the hot pack case 350, the hot pack case 350 is first received in the outer receiving box 220 and then is moved to the inner receiving box 210 after a predetermined period of time, according to the temperature of the hot pack 400.

Hereinafter, a continuous thermotherapy device according to a third embodiment of the invention will be described. Descriptions of the same components as those of the continuous thermotherapy device according to the first embodiment will be omitted.

The continuous thermotherapy device according to the third embodiment of the invention includes a hot pack 400 and a hot pack case 300.

Referring to FIGS. 1 to 5, the hot pack case 300 includes an opening 311 formed at an upper portion thereof and a case main body 310 having a hexahedral shape. The case main body 310 is formed such that an upper thickness W1 of a side surface is greater than a lower thickness W2 of the side surface. Accordingly, even if gravity is applied to the hot pack 400 for a long period of time as the hot pack 400 is continuously received in the hot pack case 300, the hot pack case 300 can prevent a pyrogen 410 of the hot pack 400 from leaning in the direction of gravity, namely, a downward direction.

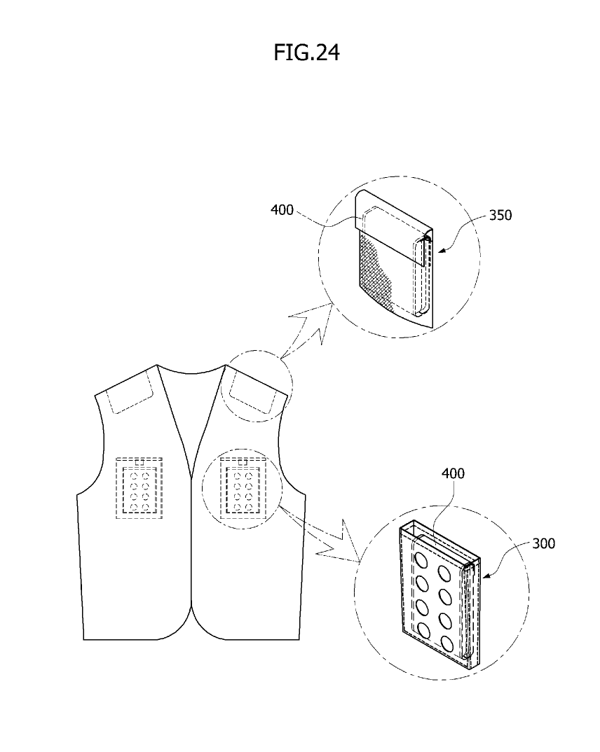

The hot pack case 300 encloses the hot pack 400, and may be received in a front receiving pocket of a vest V as shown in FIG. 24. Further, the hot pack case 300 in which the hot pack 400 is accommodated may be received in another receiving space for thermotherapy in addition to the vest V.

Plural air contact holes 315 are formed on front and rear surfaces of the hot pack case 300. An amount of external air contacting the hot pack 400 received in the hot pack case 300 may be increased by the air contact holes 315.

Since the hot pack case 300 directly receives heat from the hot pack 400 received therein, the hot pack case 300 is formed of a material having heat resistance. According to the present embodiment, the hot pack case 300 is formed of nylon.

Referring to FIGS. 6 to 8, each of the hot packs 400 includes a pyrogen 410 and a housing 420 accommodating the pyrogen 410.

The housing 420 has a shape as shown in FIG. 6. The housing 420 is formed such that a front plate 421 and a rear plate 422 may be folded.

The pyrogen 410 expands over time after exothermic reaction is initiated, in which case the folded front and rear plates 421, 422 buffer expansion of the pyrogen 410 while being unfolded according to the degree of expansion (see FIGS. 18 and 19).

However, according to the present invention, even if the pyrogen 410 expands, the foldable front and rear plates 421, 422 sufficiently buffer the expansion to prevent only the expanded portion from being convexly thickened, thereby restricting a local burn.

The housing 420 includes the front plate 421, the rear plate 422, a right plate 423, and a left plate 424.

The front plate 421 configures a front surface of the housing 420 and is formed in a rectangular shape. The rear plate 422 configures a rear surface of the housing 420 and is formed in a rectangular shape identically to the front plate 421. Upper and lower ends of the rear plate 422 are stitched to upper and lower ends of the front plate 421, respectively.

The right plate 423 configures a right surface (based on FIG. 6) of the housing 420. A front end of the right plate 423 is connected to the front plate 421 and a rear end of the right plate 423 is connected to the rear plate 422. The right plate 423 is formed such that a width of each of upper and lower ends is less than that of a central portion (see FIG. 6).

The left plate 424 faces the right plate 423 and configures a left surface of the housing 420. A front end of the left plate 424 is connected to the front plate 421 and a rear end of the left plate 424 is connected to the rear plate 422. The left plate 424 is formed such that a width of each of upper and lower ends is less than that of a central portion (see FIG. 6).

Since an internal space of the housing 420 is larger than the volume of the pyrogen 410, compact insertion of the hot pack 400 into the hot pack case 300 is realized by folding the housing 420.

For folding the housing 420, lower portions of the front and rear plates 421, 422 are folded as shown in FIG. 7. In this case, a width of a lower end of the left and right plates 424, 423 is made identical to the width of the central portion thereof. Accordingly, front and rear widths of the pyrogen 410 embedded in a lower portion of the housing 420 are made identical to those of the pyrogen 410 embedded in a central portion of the housing 420.

Next, an upper portion of each of the front and rear plates 421, 422 is folded as shown in FIG. 8. In this case, a width of an upper end of each of the left and right plates 424, 423 is made identical to the width of the central portion thereof. Accordingly, front and rear widths of the pyrogen 410 embedded in an upper end of the housing 420 are made identical to those of the pyrogen 410 embedded in the central portion of the housing 420.

The hot pack 400 is inserted in the shape shown in FIG. 8 into the hot pack case 300 (see FIG. 18). Since the hot pack 400 is compactly inserted into the hot pack case 300, an internal space of the hot pack case 300 has the same volume as that of the hot pack 400 folded as shown in FIG. 8.

The housing 420 may be unfolded or folded, as described above. With the housing unfolded, the pyrogen 410 accommodated in the housing 420 may be easily mixed and the conglomerated pyrogen 410 may also be broken into small pieces. Further, when the hot pack 400 is inserted into the hot pack case 300, an outer shape of the hot pack 400 can be changed to exactly fit the hot pack case 300 by folding the housing 420.

The continuous thermotherapy device according to the third embodiment may further include an air shielding film 500.

The air shielding film 500 encloses the hot pack 400 to prevent contact between the hot pack 400 and air. Temperature of the hot pack 400 may be adjusted through the air shielding film 500. The air shielding film 500 includes a front shielding film 510, a rear shielding film 520, a top shielding film 530, a bottom shielding film 540, a first integral shielding film 550, and a second integral shielding film 560, and each of the shielding films may be used alone or a plurality of shielding films may be simultaneously used.

Since temperature of the hot pack 400 suitable for thermotherapy ranges from 40° C. to 45° C., the hot pack 400 is enclosed by the air shielding film 500 to reach the desired temperature through the methods, as shown in FIGS. 11 to 17.

Hereinafter, a continuous thermotherapy device according to a fourth embodiment of the invention will be described. Description of the same components as those of the continuous thermotherapy device according to the first embodiment will be omitted.

The continuous thermotherapy device according to the fourth embodiment of the invention includes a hot pack 400 and a hot pack pouch 350.

Referring to FIGS. 20 to 23, each of the hot pack pouches 350 has an opening 351 formed at an upper portion thereof and is formed in a shape of a document envelope. The hot pack pouch 350 is formed of a flexible material to enclose a bent portion, such as a knee, an elbow, and the like.

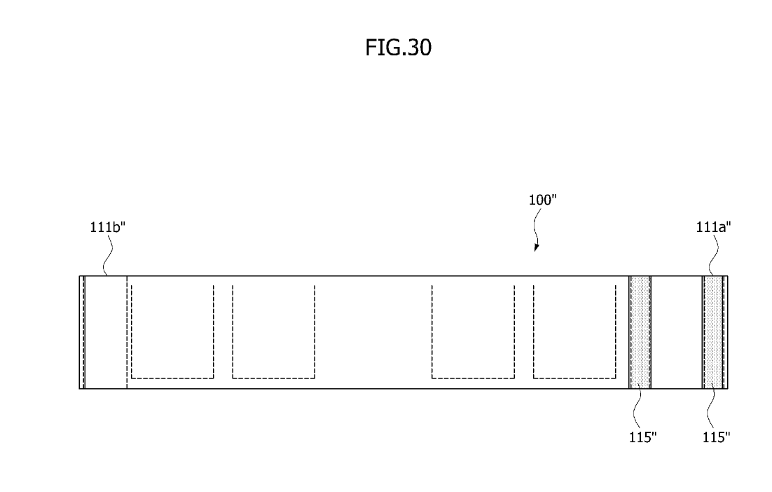

The hot pack pouch 350 encloses the hot pack 400 and may be received in a front receiving pocket of a vest V, as shown in FIG. 30. Further, the hot pack pouch 350 in which the hot pack 400 is accommodated may be utilized for thermotherapy in an eye bandage, a headband, a knee guard, a wrist guard, an ankle warmer, a thigh garter, and the like, in addition to the vest V.

In this embodiment, the hot pack pouch 350 is formed of non-woven fabrics. An amount of air contacting the hot pack 400 may be adjusted through the hot pack pouch 350 surrounding the hot pack 400 and an outer hot pack pouch 360 in addition to the air shielding film 500. A hot pack pouch additionally surrounding the hot pack 400 is referred to as the outer hot pack pouch 360, and the outer hot pack pouch 360 may also be formed of non-woven fabrics.

Referring to FIGS. 6 to 8, each of the hot packs 400 includes a pyrogen 410 and a housing 420 accommodating the pyrogen 410.

The housing 420 has a shape as shown in FIG. 6. The housing 420 is formed such that a front plate 421 and a rear plate 422 may be folded.

The pyrogen 410 expands over time after exothermic reaction is initiated, in which case the folded front and rear plates 421, 422 buffer expansion of the pyrogen 410 while being unfolded according to the degree of expansion (see FIGS. 18 and 19).

However, according to the present invention, even if the pyrogen 410 expands, the foldable front and rear plates 421, 422 sufficiently buffer the expansion to prevent only the expanded portion from being convexly thickened, thereby restricting a local burn.

The housing 420 includes the front plate 421, the rear plate 422, a right plate 423, and a left plate 424.

The front plate 421 configures a front surface of the housing 420 and is formed in a rectangular shape. The rear plate 422 configures a rear surface of the housing 420 and is formed in a rectangular shape identically to the front plate 421. Upper and lower ends of the rear plate 422 are stitched to upper and lower ends of the front plate 421, respectively.

The right plate 423 configures a right surface (based on FIG. 6) of the housing 420. A front end of the right plate 423 is connected to the front plate 421 and a rear end of the right plate 423 is connected to the rear plate 422. The right plate 423 is formed such that a width of each of upper and lower ends is less than that of a central portion (see FIG. 6).

The left plate 424 faces the right plate 423 and configures a left surface of the housing 420. A front end of the left plate 424 is connected to the front plate 421 and a rear end of the left plate 424 is connected to the rear plate 422. The left plate 424 is formed such that a width of each of upper and lower ends is less than that of a central portion (see FIG. 6).

Since an internal space of the housing 420 is larger than the volume of the pyrogen 410, compact insertion of the hot pack 400 into the hot pack case 300 is realized by folding the housing 420.

For folding the housing 420, lower portions of the front and rear plates 421, 422 are folded as shown in FIG. 7. In this case, a width of a lower end of the left and right plates 424, 423 is made identical to the width of the central portion thereof. Accordingly, front and rear widths of the pyrogen 410 embedded in a lower portion of the housing 420 are made identical to those of the pyrogen 410 embedded in a central portion of the housing 420.

Next, an upper portion of each of the front and rear plates 421, 422 is folded as shown in FIG. 8. In this case, a width of an upper end of each of the left and right plates 424, 423 is made identical to the width of the central portion thereof. Accordingly, front and rear widths of the pyrogen 410 embedded in an upper end of the housing 420 are made identical to those of the pyrogen 410 embedded in the central portion of the housing 420.

The hot pack 400 is inserted in the shape shown in FIG. 8 into the hot pack case 300 (see FIG. 18). Since the hot pack 400 is compactly inserted into the hot pack case 300, an internal space of the hot pack case 300 has the same volume as that of the hot pack 400 folded as shown in FIG. 8.

The housing 420 may be unfolded or folded, as described above. With the housing unfolded, the pyrogen 410 accommodated in the housing 420 may be easily mixed and the conglomerated pyrogen 410 may also be broken into small pieces. Further, when the hot pack 400 is inserted into the hot pack case 300, an outer shape of the hot pack 400 can be changed to exactly fit the hot pack case 300 by folding the housing 420.

The continuous thermotherapy device according to the fourth embodiment may further include an air shielding film 500.

The air shielding film 500 encloses the hot pack 400 to prevent contact between the hot pack 400 and air. Temperature of the hot pack 400 may be adjusted through the air shielding film 500. The air shielding film 500 includes a front shielding film 510, a rear shielding film 520, a top shielding film 530, a bottom shielding film 540, a first integral shielding film 550, and a second integral shielding film 560, and each of the shielding films may be used alone and a plurality of shielding films may be simultaneously used.

Since temperature of the hot pack 400 suitable for thermotherapy ranges from 40° C. to 45° C., the hot pack 400 is enclosed by the air shielding film 500 to fit the temperature through the methods as shown in FIGS. 11 to 17.

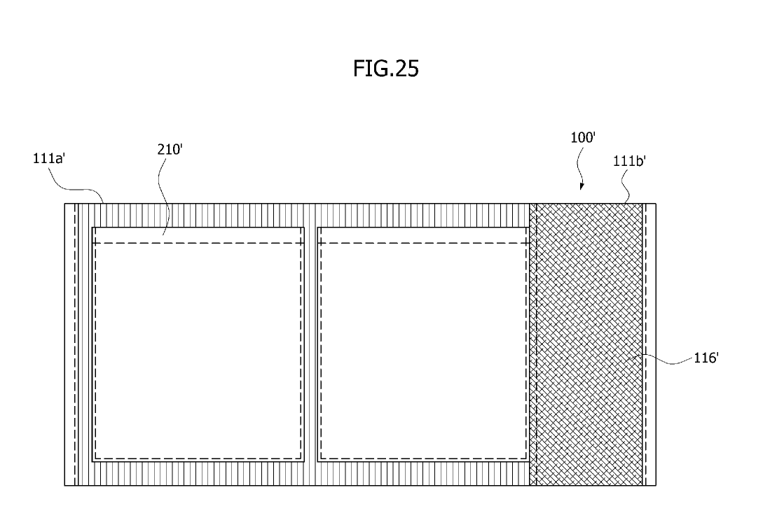

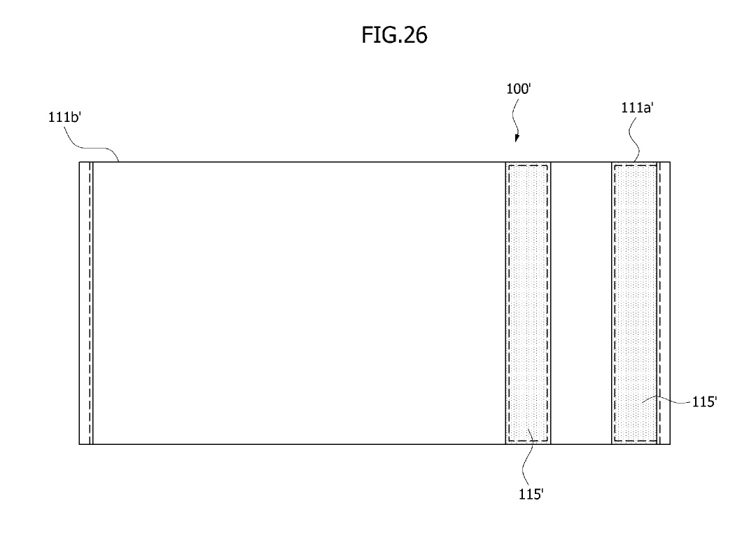

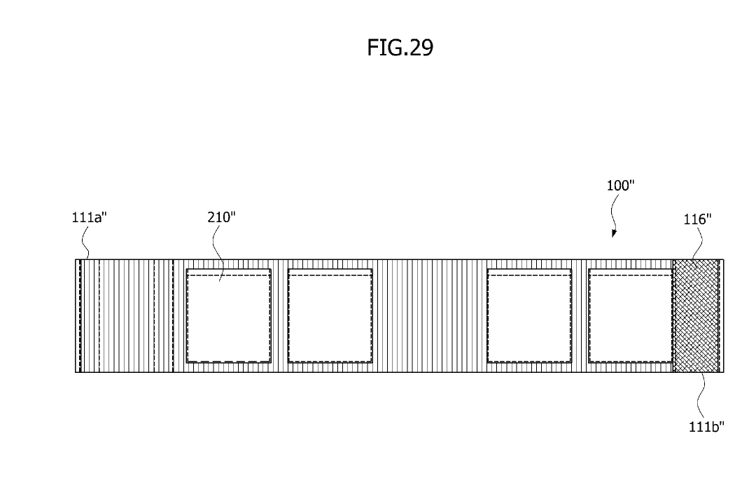

The continuous thermotherapy device according to the present invention may be modified similarly to a first modified embodiment (see FIGS. 25 to 28) and a second modified embodiment (see FIGS. 29 to 32).

The continuous thermotherapy device according to the first modified embodiment of the invention is used for thermotherapy of body parts, a diameter is not so large of which, such as the hand, wrist, forearm, and the like, and the continuous thermotherapy device according to the second modified embodiment of the invention is used for thermotherapy of body parts, a diameter is relatively large of which, such as the calf, thigh, head, neck, shoulder, and the like.

Referring to FIGS. 25 to 28, the continuous thermotherapy device according to the first modified embodiment includes a belt 100′, inner receiving boxes 210′, and hot packs 400.

The belt 100′ is formed of a flexible material such that a length of the belt 100′ may be varied. Accordingly, the belt 100′ may be attached to a body part, thereby enhancing an effect of thermotherapy. According to the present embodiment, the belt 100′ may be formed of spandex.

The inner receiving boxes 210′ are formed inside the belt 100′. The inner receiving boxes 210′ are open at upper sides thereof and the hot packs 400 or hot pack cases 300 are inserted into the inner receiving boxes 210′ through the open upper sides, respectively.

The belt 100′ is formed at one end 111a′ thereof with female Velcro tapes 115′ and at the other end 111b′ thereof with a male Velcro tape 116′. The opposite ends 111a′, 111b′ of the belt 100′ are attached to or detached from each other through attachment/detachment of the female and male Velcro tapes 115′, 116′.

A plurality of female Velcro tapes 115′ is disposed at the one end of the belt 100′ to be separated from each other. In the present embodiment, two female Velcro tapes 115′ are disposed at the one end of the belt 100′ to be separated from each other. Of course, three or more female Velcro tapes 115′ may be disposed to be separated from each other.

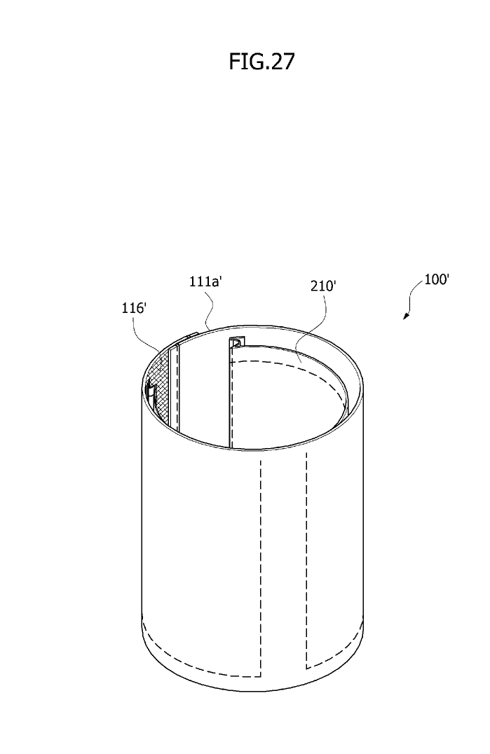

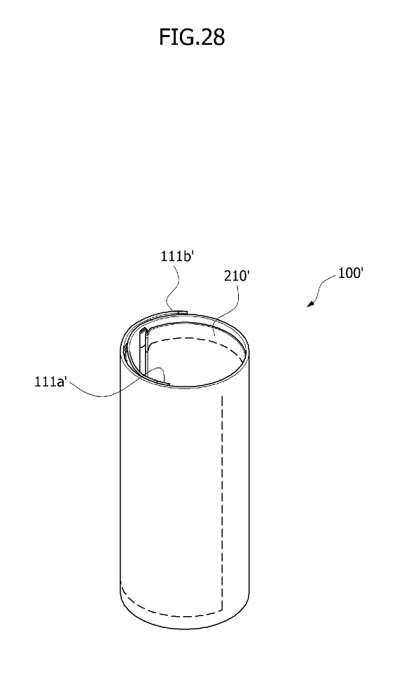

The belt 100′ forms a ring shape by attaching the male Velcro tape 116′ to the female Velcro tapes 115′ (see FIGS. 27 and 28). A peripheral length of the ring shape formed by the belt 100′ varies depending on the location of the female Velcro tape 115′ to which the male Velcro tape 116′ is attached. Accordingly, the peripheral length of the belt 100′ may be adjusted to fit a periphery of a user's body part, thereby optimizing the belt 100′ for the user's body size.

FIG. 27 shows a state in which the male Velcro tape 116′ is attached to an outside female Velcro tape 115′, and FIG. 28 shows a state in which the male Velcro tape 116′ is attached to an inside female Velcro tape 115′. The periphery of the belt 100′ shown in FIG. 27 is greater than that of the belt 100′ shown in FIG. 28.

Referring to FIGS. 29 to 32, the continuous thermotherapy device according to the second modified embodiment of present invention includes a belt 100″, inner receiving boxes 210″, and hot packs 400. The belt 100″ of the second modified embodiment is formed longer than the belt 100′ of the first modified embodiment and thus, is utilized for thermotherapy of a body part having a large periphery relative to the body part on which the belt 100′ of the first modified embodiment is worn.

The belt 100″ is formed of a flexible material such that a length of the belt 100″ may be varied. Accordingly, the belt 100″ may be attached to a body part, thereby enhancing an effect of thermotherapy. According to the present embodiment, the belt 100″ may be formed of spandex.

The inner receiving boxes 210″ are formed inside the belt 100″. The inner receiving boxes 210″ are open at upper sides thereof and the hot packs 400 or hot pack cases 300 are inserted into the inner receiving boxes 210″ through the open upper sides, respectively. In this embodiment, the belt 100″ is provided with four inner receiving boxes. Specifically, the belt 100″ is at one end thereof with two inner receiving boxes 210″ and at the other end thereof with two inner receiving boxes 210″.

The belt 100″ is provided at one end 111a″ thereof with female Velcro tapes 115″ and at the other end 111b″ thereof with a male Velcro tape 116″. The opposite ends 111a″ and 111b″ of the belt 100″ are attached to or detached from each other through attachment/detachment of the female and male Velcro tapes 115″ and 116″.

A plurality of female Velcro tapes 115″ is disposed at the one end of the belt 100″ to be separated from each other. According to the present embodiment, two female Velcro tapes 115″ are disposed at the one end of the belt 100″ to be separated from each other. Of course, three or more female Velcro tapes 115″ may be disposed to be separated from each other.

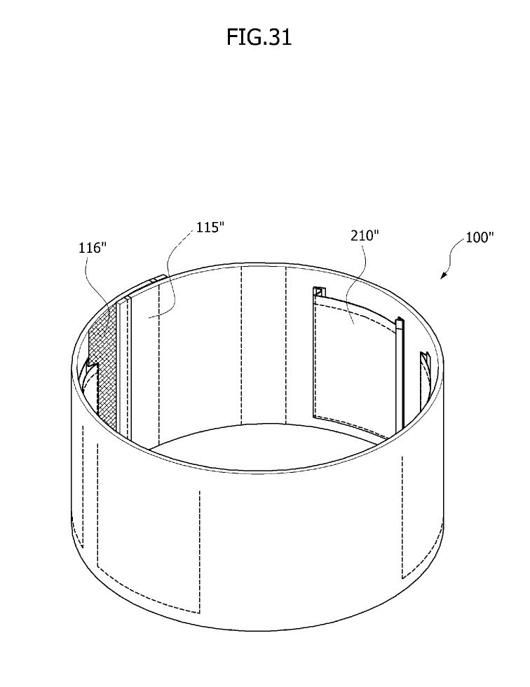

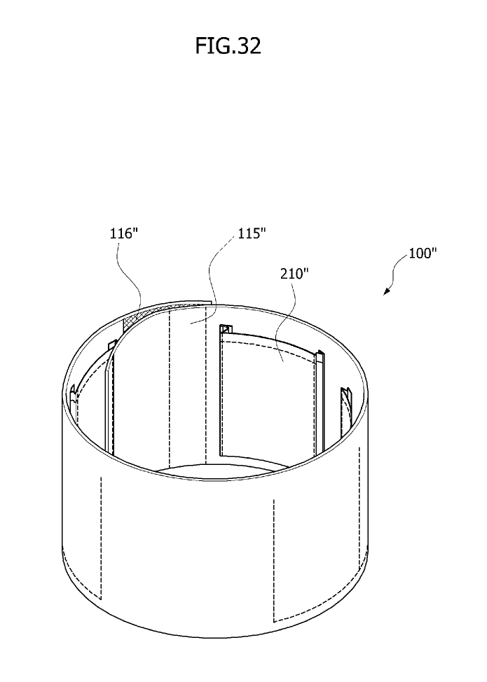

The belt 100″ forms a ring shape by attaching the male Velcro tape 116″ to the female Velcro tape 115″ (see FIGS. 31 and 32). A peripheral length of the ring shape formed by the belt 100″ varies depending on the location of the female Velcro tape 115″ to which the male Velcro tape 116′ is attached. Accordingly, the peripheral length of the belt 100″ may be adjusted to fit a periphery of a user's body part, thereby optimizing the belt 100″ for the user's body size.

FIG. 31 shows a state in which the male Velcro tape 116″ is attached to an outside female Velcro tape 115″, and FIG. 32 shows a state in which the male Velcro tape 116″ is attached to an inside female Velcro tape 115″. The periphery of the belt 100″ shown in FIG. 31 is greater than that of the belt 100″ shown in FIG. 32.

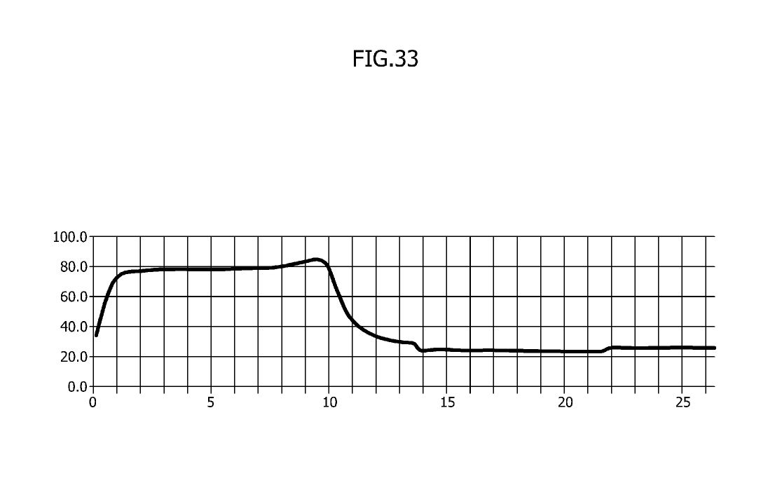

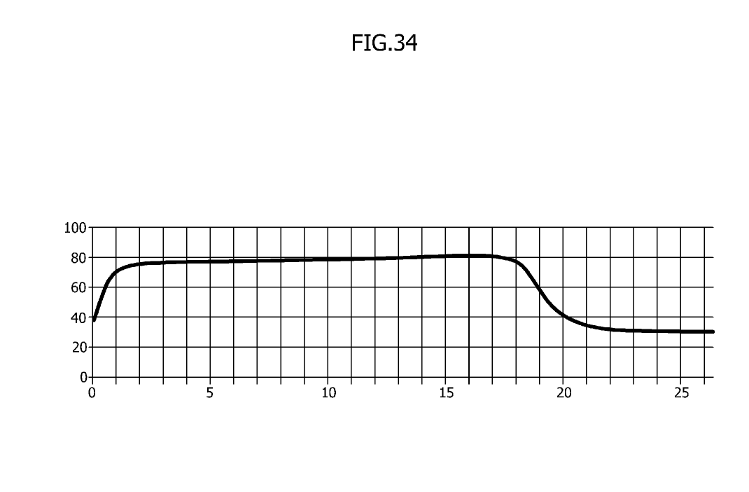

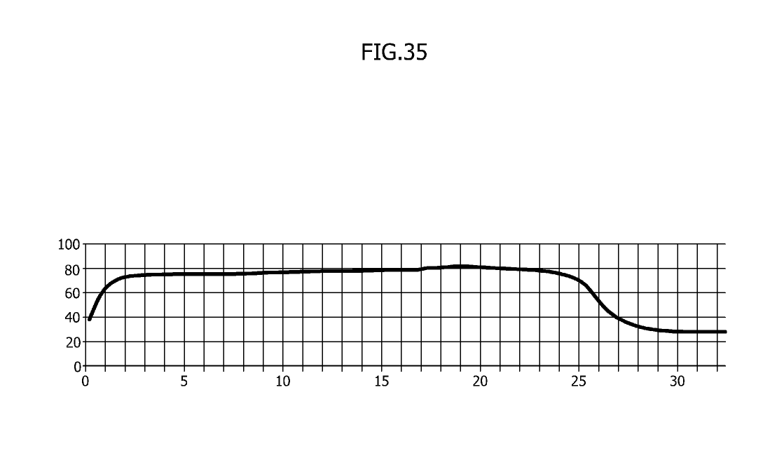

FIGS. 33 to 35 are graphs showing a change in temperature of a hot pack, in which the horizontal axis denotes time (h) and the vertical axis denotes temperature (° C.).

Although some embodiments have been described herein, it should be understood by those skilled in the art that various modifications, changes, and alterations can be made without departing from the spirit and scope of the invention. Therefore, the scope of the invention should be limited only by the accompanying claims and equivalents thereof.

1-25. (canceled)

26. A continuous thermotherapy device comprising:
a belt;
a receiving box provided to the belt;
a hot pack case received in the receiving box; and
a hot pack inserted into the hot pack case,
wherein the hot pack comprises: a pyrogen; and a housing receiving the pyrogen therein and having an internal space larger than a volume of the pyrogen such that the pyrogen is mixed within the internal space.

27. The continuous thermotherapy device according to claim 26, wherein the housing comprises:
a front plate;
a rear plate having the same shape as the front plate and stitched at upper and lower ends thereof to upper and lower ends of the front plate, respectively;
a right plate connected at a front end thereof to the front plate and at a rear end thereof to the rear plate; and
a left plate having the same shape as the right plate and facing the right plate, the left plate being connected at a front end thereof to the front plate and at a rear end thereof to the rear plate,
wherein a width of each of upper and lower ends of the left and right plates is smaller than a width of a central portion of each of the left and right plates.

28. The continuous thermotherapy device according to claim 27, wherein the front and rear plates are foldable and, when lower portions of the front and rear plates are folded, the width of the lower end of each of the left and right plates are the same as that of the central portion of each of the left and right plates.

29. The continuous thermotherapy device according to claim 26, wherein the receiving box comprises at least one of an inner receiving box open at an upper side thereof and disposed inside the belt, and an outer receiving box open at an upper side thereof and disposed outside the belt.

30. The continuous thermotherapy device according to claim 29, wherein Velcro tapes are provided to an inner surface of an upper end of the receiving box and to one side surface of the belt facing the inner surface of the upper end of the receiving box, respectively, and the upper end of the receiving box is detachably coupled to the belt through attachment/detachment of the Velcro tapes.

31. The continuous thermotherapy device according to claim 26, wherein the hot pack case comprises: a case main body that has an upper opening and is formed in a hexahedral shape, an upper thickness of a side surface of the case main body being larger than a lower thickness of the side surface of the case main body.

32. The continuous thermotherapy device according to claim 31, wherein the case main body is formed with an air contact hole.

33. The continuous thermotherapy device according to claim 26, further comprising:
an air shielding film surrounding the hot pack such that an amount of air contacting the hot pack is adjusted,
wherein the air shielding film comprises at least one of a front shielding film surrounding a front surface of the hot pack; a rear shielding film surrounding a rear surface of the hot pack; a top shielding film surrounding a top surface of the hot pack; a bottom shielding film surrounding a bottom surface of the hot pack; a first integral shielding film surrounding the front, bottom and rear surfaces of the hot pack; and a second integral shielding film surrounding the front, top, and rear surfaces of the hot pack.

34. A continuous thermotherapy device comprising:
a belt;
a receiving box provided to the belt;
a hot pack pouch received in the receiving box and formed of a flexible material to enclose a bent portion; and
a hot pack inserted into the hot pack pouch,
wherein the hot pack comprises:
a pyrogen; and
a housing receiving the pyrogen therein and having an internal space larger than a volume of the pyrogen such that the pyrogen is mixed within the internal space.

35. The continuous thermotherapy device according to claim 34, wherein the housing comprises:
a front plate;
a rear plate having the same shape as the front plate and stitched at upper and lower ends thereof to upper and lower ends of the front plate, respectively;
a right plate connected at a front end thereof to the front plate and at a rear end thereof to the rear plate; and
a left plate having the same shape as the right plate and facing the right plate, the left plate being connected at a front end thereof to the front plate and at a rear end thereof to the rear plate,
wherein a width of each of upper and lower ends of the left and right plates is smaller than a width of a central portion of each of the left and right plates.

36. A continuous thermotherapy device comprising:
a hot pack; and
a hot pack case receiving the hot pack,
wherein the hot pack case comprises a case main body having an upper opening and formed in a hexahedral shape,
wherein the hot pack comprises:
a pyrogen; and
a housing receiving the pyrogen therein and having an internal space larger than a volume of the pyrogen such that the pyrogen is mixed within the internal space.

37. The continuous thermotherapy device according to claim 36, wherein the housing comprises:
a front plate;
a rear plate having the same shape as the front plate and stitched at upper and lower ends thereof to upper and lower ends of the front plate, respectively;
a right plate connected at a front end thereof to the front plate and at a rear end thereof to the rear plate; and
a left plate having the same shape as the right plate and facing the right plate, the left plate being connected at a front end thereof to the front plate and at a rear end thereof to the rear plate,
wherein a width of each of upper and lower ends of the left and right plates is smaller than a width of a central portion of each of the left and right plates.

38. The continuous thermotherapy device according to claim 36, wherein An upper thickness of a side surface of the case main body is larger than a lower thickness of the side surface of the case main body, and the case main body is formed with an air contact hole.

39. The continuous thermotherapy device according to claim 36, further comprising:
an air shielding film surrounding the hot pack such that an amount of air contacting the hot pack is adjusted,
wherein the air shielding film comprises at least one of a front shielding film surrounding a front surface of the hot pack; a rear shielding film surrounding a rear surface of the hot pack; a top shielding film surrounding a top surface of the hot pack; a bottom shielding film surrounding a bottom surface of the hot pack; a first integral shielding film surrounding the front, bottom and rear surfaces of the hot pack; and a second integral shielding film surrounding the front, top, and rear surfaces of the hot pack.

40. A continuous thermotherapy device comprising:
a hot pack; and
a hot pack pouch receiving the hot pack and is formed of a flexible material to enclose a bent portion,
wherein the hot pack comprises:
a pyrogen; and
a housing receiving the pyrogen therein and having an internal space larger than a volume of the pyrogen such that the pyrogen is mixed within the internal space.

41. The continuous thermotherapy device according to claim 40, wherein the housing comprises:
a front plate;
a rear plate having the same shape as the front plate and stitched at upper and lower ends thereof to upper and lower ends of the front plate, respectively;
a right plate connected at a front end thereof to the front plate and at a rear end thereof to the rear plate; and
a left plate having the same shape as the right plate and facing the right plate, the left plate being connected at a front end thereof to the front plate and at a rear end thereof to the rear plate,
wherein a width of each of upper and lower ends of the left and right plates is smaller than a width of a central portion of each of the left and right plates.

42. The continuous thermotherapy device according to claim 40, further comprising:
an outer hot pack pouch receiving the hot pack pouch therein such that an amount of air contacting the hot pack is adjusted.

43. The continuous thermotherapy device according to claim 40, further comprising:
an air shielding film surrounding the hot pack such that an amount of air contacting the hot pack is adjusted,
wherein the air shielding film comprises at least one of a front shielding film surrounding a front surface of the hot pack; a rear shielding film surrounding a rear surface of the hot pack; a top shielding film surrounding a top surface of the hot pack; a bottom shielding film surrounding a bottom surface of the hot pack; a first integral shielding film surrounding the front, bottom and rear surfaces of the hot pack; and a second integral shielding film surrounding the front, top, and rear surfaces of the hot pack.

44. A continuous thermotherapy device comprising:
a belt;
an inner receiving box disposed inside the belt; and
a hot pack inserted into the inner receiving box,
wherein the hot pack comprises: a pyrogen; and a housing receiving the pyrogen therein and having an internal space larger than a volume of the pyrogen such that the pyrogen is mixed within the internal space.

45. The continuous thermotherapy device according to claim 44, wherein the housing comprises:
a front plate;
a rear plate having the same shape as the front plate and stitched at upper and lower ends thereof to upper and lower ends of the front plate, respectively;
a right plate connected at a front end thereof to the front plate and at a rear end thereof to the rear plate; and
a left plate having the same shape as the right plate and facing the right plate, the left plate being connected at a front end thereof to the front plate and at a rear end thereof to the rear plate,
wherein a width of each of upper and lower ends of the left and right plates is smaller than a width of a central portion of each of the left and right plates, and
wherein the belt is formed at one end thereof with a female Velcro tape and at the other end thereof with a male Velcro tape, the opposite ends of the belt being attached to or detached from each other through attachment/detachment of the female and male Velcro tapes.
